(19) United States
(12) Patent Application Publication
(10) Pub. No.: US 2022/0257915 A1
(43) Pub. Date: Aug. 18, 2022

(54) Skin Protection Device

(71) Applicant: Katy Hamilton, Santa Barbara, CA (US)

(72) Inventor: Katy Hamilton, Santa Barbara, CA (US)

(21) Appl. No.: 17/667,060

(22) Filed: Feb. 8, 2022

Related U.S. Application Data
(60) Provisional application No. 63/150,537, filed on Feb. 17, 2021.

(51) Int. Cl. A61M 35/00

(52) U.S. Cl. CPC A61M 35/003; A61M 35/10; A61M 2207/00; A61M 2205/0238; A61M 2209/088; A61M 2210/0618; A61M 2210/083; A61M 2210/086; A61M 2210/1021; A61M 2210/0606

(57) ABSTRACT

The present invention relates to a skin protection device primarily comprised of a body with a top surface and a bottom surface, and a backing sheet. The body is preferably flexible and is manufactured from a biodegradable, waterproof, and UV-resistant/UV-blocking material that is plastic, fabric, or latex-based. In addition, the body may be infused with a plurality of zinc particles/compounds to protect against sun/UV damage and may have differing levels of SPF protection. The body is preferably colored to resemble the skin shade or tone of the user and has an adhesive bottom surface that can adhere to a user's skin. The body may be appropriately shaped in any conventional or non-conventional shape that may or may not mimic the shape of the body part it is being applied to.

### CROSS-REFERENCE TO RELATED APPLICATION

The present application claims priority to, and the benefit of, U.S. Provisional Application No. 63/150,537, which was filed on Feb. 17, 2021 and is incorporated herein by reference in its entirety.

### FIELD OF THE INVENTION

The present invention relates generally to the field of skin protection. More specifically, the present invention relates to a skin protection device primarily comprised of a body with a top surface and a bottom surface, and a backing sheet. The body is preferably flexible and is manufactured from a biodegradable, waterproof, and UV-resistant/UV-blocking material that is plastic, fabric, or latex-based. In addition, the body may be infused with a plurality of zinc particles/compounds to protect against sun/UV damage and may have differing levels of SPF protection. The body is preferably colored to resemble the skin shade of the user, and has an adhesive bottom surface that can adhere to a user's skin. Accordingly, the present disclosure makes specific reference thereto. Nonetheless, it is to be appreciated that aspects of the present invention are also equally applicable to other like applications, devices and methods of manufacture.

### BACKGROUND

When spending a significant amount of time outdoors, an individual's skin can become overexposed to sunlight. Further, it is well-documented that human skin is sensitive to sunlight and artificial light containing radiation of wavelengths between about 290 nanometers (nm) and 400 nm. Ultraviolet radiation of wavelengths between about 290 nm and 320 nm (UV-B region) has been known to rapidly produce damaging effects on the skin, including reddening or erythema, edema, blistering or other skin eruptions in more severe cases. Prolonged or chronic exposure to radiation in this wavelength range has been associated with serious skin conditions, such as actinic keratoses and carcinomas. In recent years, concern has also been expressed regarding ultraviolet radiation of wavelengths above 320 nm (UV-A region) and the adverse effects of such radiation on human skin. The radiation between 320 and 400 nm also contributes to the premature aging of the skin. In addition, recent studies indicate that chronic sun exposure limits the immuno-response of the skin. There is also evidence that a tan will offer some protection against burning but is quite ineffectual against other types of solar damage.

Growing public awareness that the enjoyment of outdoor activities must go hand-in-hand with adequate sun protection has led to an unprecedented growth in the area of sunscreen products. A desirable sunscreen product should have the following attributes: protection in both the UV-A and UV-B ultraviolet radiation ranges; maintenance of coverage, i.e., waterproof and perspiration proof; application and use convenience, i.e., ease of application, invisibility, non-staining and non-greasy; and freedom from irritation as a result of its ingredients, in particular its active sunscreen ingredients.

In addition, sunscreen is often stored in bulky containers and bottles. These containers and bottles occupy an undesirable amount of space. Therefore, transporting the sunscreen when traveling or spending a significant time outside can be cumbersome for the individual, especially when they possess limited storage space.

Therefore, there exists a long-felt need in the art for an improved device that protects the skin of a user from the sun's rays. There also exists a long-felt need in the art for a skin protection device that protects a user's skin from the sun but does not require a significant amount of storage space. Further, there exists a long-felt need in the art for a skin protection device that does not involve a messy application process.

The subject matter disclosed and claimed herein, in one embodiment thereof, comprises a skin protection device. The device is primarily comprised of a body with a top surface and a bottom surface and a backing sheet. The body is preferably flexible and is manufactured from a biodegradable, waterproof, and UV-resistant/UV-blocking material that is plastic, fabric or latex-based. In addition, the body may be infused with a plurality of zinc particles/compounds to protect against sun/UV damage and may have differing levels of SPF protection. The body is preferably colored to resemble the skin shade of the user and has an adhesive bottom surface that can adhere to a user's skin. The body may be appropriately shaped in any conventional or non-conventional shape, that may or may not mimic the shape of the body part it is being applied to, such as but not limited to, the nose, the face, the shoulders, the chest, or other suitable areas, such as but not, limited to: arms, legs, feet, hands, back, stomach, etc.

In this manner, the skin protection device of the present invention accomplishes all of the forgoing objectives and provides an improved device that protects the skin of a user from the sun's rays in a manner similar to sunscreen. However, the device does not require a significant amount of storage space and does not involve a messy application process. Yet, in doing so the device still offers the SPF protection found in existing sunscreens known in the art. In this manner, the skin protection device overcomes the limitations of existing sunscreens and sunscreen application methods known in the art.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a skin protection device. The device is primarily comprised of a body with a top surface and a bottom surface, and a backing sheet. Specifically, the body comprises a top surface of non-woven, random-spun, synthetic fiber fabric material capable of blocking harmful ultra-violet (UV) radiation; and a bottom layer of skin-compatible, pressure sensitive, non-toxic, hypo-allergenic, non-irritating adhesive material applied to the underside of the non-woven material by any one of many well-known methods.

Furthermore, the body is preferably flexible and is manufactured from a biodegradable, waterproof and UV-resistant/UV-blocking material that is plastic, fabric or latex-based. In addition, the body may be infused with a plurality of zinc particles/compounds to protect against sun/UV damage and may have differing levels of SPF protection. The body may also have a plurality of perforations that may be circular and aid in breathability and/or may be linear and used to attach multiple devices together (i.e., in a sequential fashion), such that they can be torn apart from one another when ready for use. The body also preferably has the color of any skin tone or shade, such that when applied to the skin of a user, the device blends in with the skin tone of the user and is virtually invisible once applied. In another embodiment, the body may comprise colors that contrast the skin tone of the user as well.

In differing embodiments, the shape of the body may differ and may be based on the intended body area to which the device is applied. Accordingly, the body may be appropriately-shaped in any conventional or non-conventional shape. The shape of the body may mimic the shape of the body part it is being applied to such as, but not limited to, the nose, the face, the shoulders, the chest, or other suitable areas such as, but not limited to, the arms, legs, feet, hands, back, stomach, etc.

When not in use, the device or multiple devices can be stored on a biodegradable backing sheet. Further, the bottom surface of the body has a skin-safe and waterproof adhesive that allows the device to remain on the skin of the user and also allows it to attach to the backing sheet when not in use. Multiple devices can be stored on the top surface of the backing sheet, such that a single device or multiple devices can then be pulled from the backing sheet when needed for use. In this manner, a plurality of devices can be conveniently stored in a flat manner that takes up minimal storage space.

Accordingly, the skin protection device of the present invention is particularly advantageous as it protects the skin of a user from the sun's rays but does not require a significant amount of storage space. Further, the device does not involve a messy application process, but still offers the SPF protection found in existing sunscreens known in the art. In this manner, the skin protection device overcomes the limitations of existing sunscreens and sunscreen application methods known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of the skin protection device of the present invention in accordance with the disclosed architecture;

FIG. 2 illustrates a perspective view of one potential embodiment of multiple skin protection devices of the present invention worn by a user in accordance with the disclosed architecture; and

FIG. 3 illustrates a perspective view of one potential embodiment of multiple skin protection devices of the present invention in use in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for an improved device that protects the skin of a user from the sun's rays. There also exists a long-felt need in the art for a skin protection device that protects a user's skin from the sun but does not require a significant amount of storage space. Further, there exists a long-felt need in the art for a skin protection device that does not involve a messy application process.

The present invention, in one exemplary embodiment, is comprised of a skin protection device that is primarily comprised of a body with a top surface and a bottom surface, and a backing sheet. The body is preferably flexible and is manufactured from a biodegradable, waterproof, and UV-resistant/UV-blocking material that is plastic, fabric or latex-based. In addition, the body may be infused with a plurality of zinc particles/compounds to protect against sun/UV damage. The body material may also have differing levels of SPF protection. In addition, the body may have a plurality of perforations that may be circular and aid in breathability and/or may be linear and used to attach multiple devices together (i.e., in a sequential fashion), such that they can be torn apart from one another when ready for use. In a preferred embodiment, the material of the body is the color of any skin shade or tone, such that when applied to the skin of a user, the device blends with the skin tone of the user. Although, in another embodiment, the body may comprise colors that contrast to the skin tone of the user.

The shape of the body may differ and may be based on the intended body area to which the device will be applied to. Accordingly, the body may be appropriately shaped in any conventional or non-conventional shape to mimic or contrast the shape of the body part it is being applied to, such as but not limited to, the nose, the face, the shoulders, the chest or other suitable areas such as, but not limited to, the arms, legs, feet, hands, back, stomach, etc.

When not in use, the device or multiple devices can be stored on a biodegradable backing sheet. The bottom surface of the body has a skin-safe and waterproof adhesive that allows the device to remain on the skin of the user and also allows it to attach to the backing sheet when not in use. Multiple devices can be stored on the top surface of the backing sheet, such that a single device or multiple devices can be pulled from the backing sheet when needed for use. In this manner, a plurality of devices can be conveniently stored in a flat manner that takes up minimal storage space.

Accordingly, the skin protection device of the present invention is particularly advantageous as it protects the skin of a user from the sun's rays but does not require a significant amount of storage space. Further, the device does not involve a messy application process, but still offers the SPF protection found in existing sunscreens known in the art. In this manner, the skin protection device overcomes the limitations of existing sunscreens and sunscreen application methods known in the art

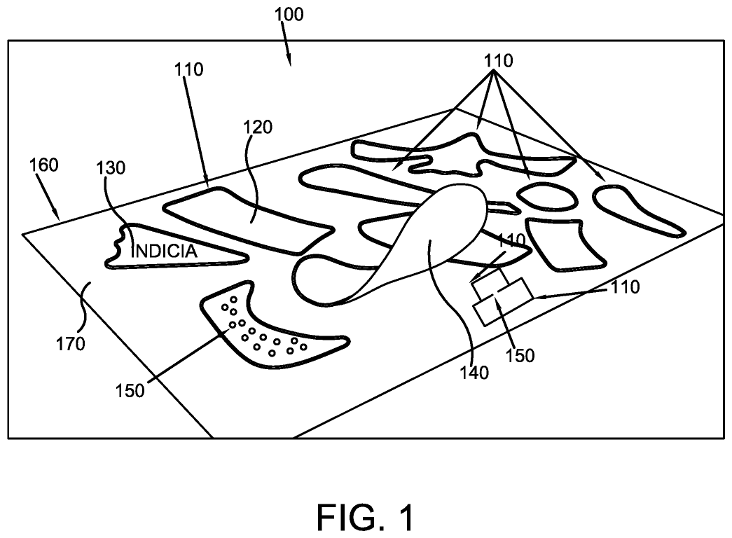

Referring initially to the drawings, FIG. 1 illustrates a perspective view of one embodiment of a skin protection device of the present invention. The device 100 is primarily comprised of a body 110 with a top surface 120 and a bottom surface 140, and a backing sheet 160. Specifically, the body 110 comprises a top surface 120 of non-woven, random-spun (or spunbonded) synthetic fiber fabric material capable of blocking harmful ultra-violet (UV) radiation; and a bottom layer 140 of a relatively thin layer of skin-compatible, pressure-sensitive, non-toxic, hypo-allergenic, non-irritating adhesive material applied to the underside of the non-woven material by any one of many well-known methods. The body 110 is mounted, for pre-use purposes (printing, packaging, storage, etc.), on release paper or a backing sheet 160 of any well-known type (silicone release paper, wax paper, etc.).

The body 110 is preferably flexible and is manufactured from a biodegradable, waterproof, and UV-resistant/UV-blocking material that is plastic, fabric and/or latex-based, or any other suitable material as is known in the art. Preferably, the top surface 120 of the body 110 is comprised of a non-woven, synthetic fiber fabric material layer formed of random-spun or spunbonded polyester and polyamide fibers and mixed polyamide/polyester fibers (fabric weight of ¼ to 2 oz/yd² and thickness of ½ to 4 mils). Alternatively, such layer may comprise fabrics of spun-laced polyester fibers, polyester/rayon blend fibers and polyester/cellulosic blend fibers. These latter materials (thickness range of 10-40 mils) are soft, strong, conformable and light weight (1-4 oz/yd²), impervious and printable, and (with appropriate coloring) may present a skin-like appearance. Further, the fabrics would exhibit a good wet and dry strength and contain no chemical binders to irritate skin areas. Another fabric is a spunbonded polyester fiber which has been coated with an acrylic emulsion to impart superior printing characteristics, i.e., high acceptability to silk screen printing inks. Additional materials may include bamboo and plastic free rice paper.

The body 110 can further be infused with a plurality of titanium dioxide and zinc oxide, or zinc particles/compounds, etc., or any other suitable compounds as is known in the art to protect against sun/UV exposure and/or soothing compounds, such as aloe vera, Vitamin E, or any other suitable compounds as are known in the art. In differing embodiments, the material of the body 110 may have differing levels of SPF protection. Said levels may include, but are not limited to: SPF 2, SPF 5, SPF 8, SPF 10, SPF 15, SPF 20, SPF 25, SPF 30, SPF 35, SPF 45, SPF 50, SPF 100, or any other suitable SPF protection level as is known in the art.

In addition, the body 110 comprises a plurality of perforations 150. The perforations 150 may be circular and located on the top surface 120 to aid in breathability of the body 110 material, and/or the perforations 150 may also be linear and used to attach multiple devices 100 together (i.e., in a sequential fashion), such that they can be torn apart from one another when ready for use. The top surface 120 may further be comprised of a plurality of indicia 130, such as but not limited to: patterns, logos, emblems, images, symbols, designs, letters, words, characters, animals, advertisements, brands, etc., or any other suitable indicia as is known in the art. In differing embodiments, the body 110 may also be dyed any suitable color known in the art. Specifically, the body 110 can have the color of any skin shade or tone known in the art, such that when applied to the skin of a user 10, the device 100 blends in with the skin tone of the user 10 and once applied to the skin, the device 100 appears almost completely invisible. In another embodiment, the body 110 may have colors that contrast the skin tone of the user 10.

Further, the device 100 is comprised of a biodegradable backing sheet 160, wherein once the body 110 is removed from the backing sheet 160, the body 110 can be placed on a user 10 and secured. Specifically, the bottom surface 140 of the body 110 has a skin-safe and waterproof adhesive that allows the device 100 to remain on the skin of the user 10, until the user 10 wishes to remove it. Multiple devices 100 can also be stored on the top surface 170 of the backing sheet 160 via the adhesive bottom surface 140, wherein each device 100 can be pulled from the backing sheet 160 when ready for use. In this manner, a plurality of devices 100 can be conveniently stored in a flat manner that takes up minimal storage space.

Additionally, the bottom surface 140 of the body 110 typically comprises a skin-compatible, non-toxic, hypo-allergenic, non-irritating adhesive, which is applied to the underside of the body 110. The adhesive may be a pressure-sensitive aqueous based or solvent-based adhesive formed of polymerized or copolymerized acrylic or a vinyl polymer or a hot melt adhesive of acrylic-based or rubber-based type. Preferably, the adhesive is an emulsion-type (aqueous-based) acrylic adhesive which leaves no residue on the skin surface after removal of the device 100 because such adhesive has greater cohesion to itself and adhesion to the body 110 than its adhesion to the skin surface. Because of the foregoing cohesion/adhesion characteristics of the adhesive, the device 100 may be reused, i.e., reapplied to a skin surface multiple times.

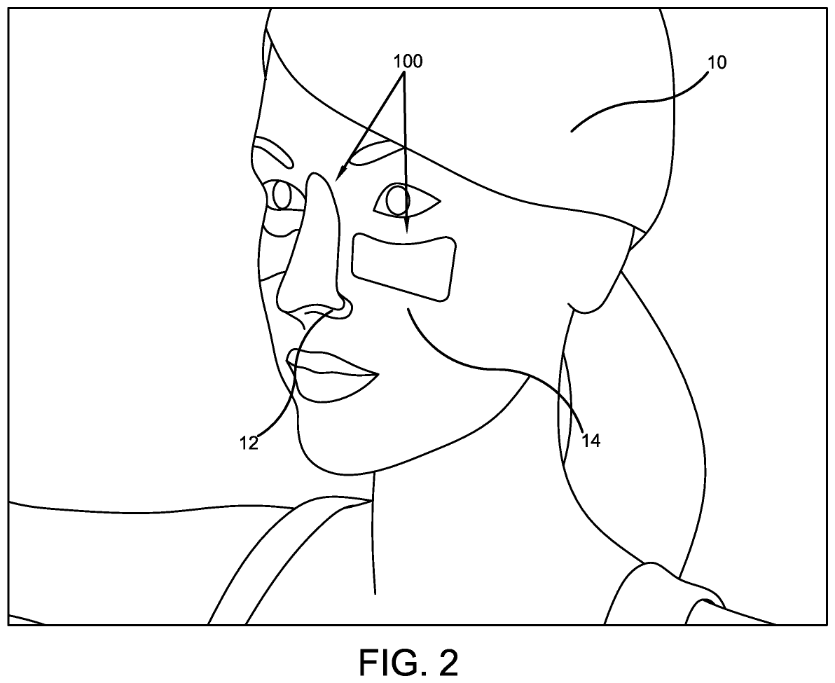

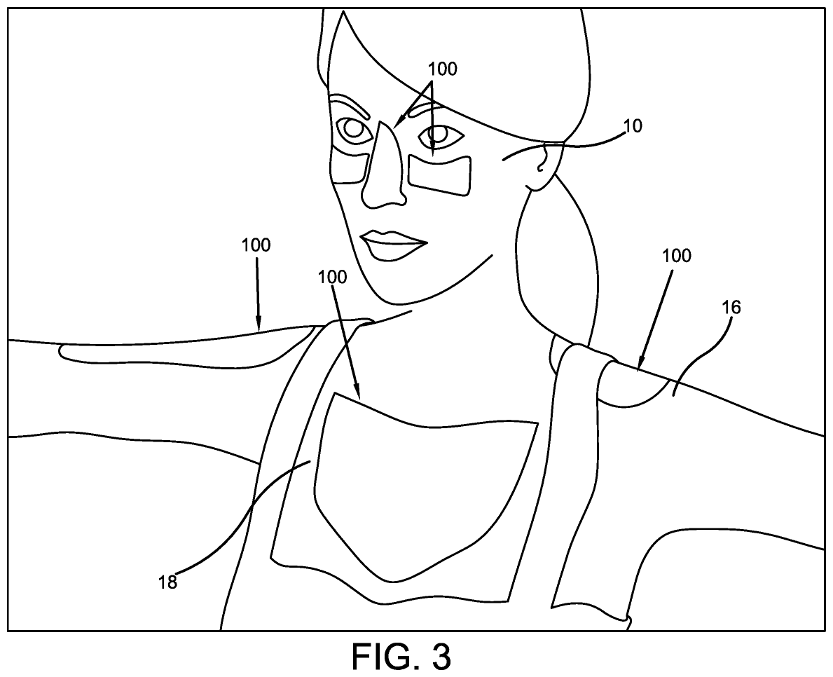

FIG. 2 and FIG. 3 illustrate a perspective view of one embodiment of multiple skin protection devices of the present invention worn by a user. In differing embodiments, the body 110 shape may differ based on the intended body area to which the device 100 is applied. As such, the body 110 may be shaped in any conventional or non-conventional shape depending on the needs and/or wants of a user. The shape of the body 110 may or may not further mimic the shape of the body part it is being applied to and can be as large as or as small as a user desires. The device 100 can also be pre-cut into specific shapes, or may be acquired as one continuous sheet, allowing a user to cut as much as they need, depending on their wants and/or needs. As such, the device 100 can be applied to specific body areas, such as but not limited to: the nose 12, the face 14, the shoulders 16, the chest 18, or other suitable areas such as, but not limited to, the arms, legs, feet, hands, back, stomach, etc., or any other suitable body area as is known in the art.

Furthermore, the top surface 120 may comprise a plurality of indicia 130 such as, but not limited to, patterns, logos, emblems, images, symbols, designs, letters, words, characters, animals, advertisements, brands, etc., or any other suitable indicia as is known in the art. In differing embodiments, the body 110 may also be dyed any suitable color known in the art. Specifically, the body 110 can have the color of any skin shade or tone known in the art, such that when applied to the skin of a user 10, the device 100 blends in with the skin tone of the user 10 and once applied to the skin, the device 100 appears almost completely invisible. In another embodiment, the body 110 may have colors that contrast the skin tone of the user 10.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “skin-protection device” and “device” are interchangeable and refer to the skin protection device 100 of the present invention.

Notwithstanding the forgoing, the skin protection device 100 of the present invention and its various components can be of any suitable size and configuration as is known in the art without affecting the overall concept of the invention, provided that they accomplish the above-stated objectives. One of ordinary skill in the art will appreciate that the size, configuration and material of the skin protection device 100 as shown in FIGS. 1-3 is for illustrative purposes only, and that many other sizes and shapes of the skin protection device 100 are well within the scope of the present disclosure. Although the dimensions of the skin protection device 100 are important design parameters for user convenience, the skin protection device 100 may be of any size, shape and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A skin protection device comprising:
a body comprised of a top surface and an adhesive bottom surface that is applied to a body of a user; and
a backing sheet.

2. The skin protection device of claim 1, wherein the body is flexible and is manufactured from a biodegradable, waterproof, and UV-resistant or UV-blocking material.

3. The skin protection device of claim 1, wherein the body is infused with a plurality of a titanium dioxide, zinc oxide or zinc particles to protect the user against a UV exposure.

4. The skin protection device of claim 1, wherein the body has a specific level of SPF protection.

5. The skin protection device of claim 1, wherein the body is stored on the backing sheet and then peeled from the backing sheet prior to application to the body of the user.

6. A skin protection device comprising:
a flexible body comprised of a top surface having a plurality of perforations therein and a skin-safe, waterproof, adhesive bottom surface that is configured to be applied to a body of a user; and
a biodegradable backing sheet.

7. The skin protection device of claim 6, wherein the top surface is comprised of a non-woven, random-spun synthetic fiber fabric material capable of blocking a harmful ultra-violet (UV) radiation.

8. The skin protection device of claim 6, wherein the flexible body is infused with a plurality of titanium dioxide, zinc oxide or zinc particles to protect the body of the user against a UV exposure.

9. The skin protection device of claim 6, wherein the flexible body is comprised of a SPF protection, wherein said SPF protection is a select one of a SPF 2, a SPF 5, a SPF 8, a SPF 10, a SPF 15, a SPF 20, a SPF 25, a SPF 30, a SPF 35, a SPF 45, a SPF 50 or a SPF 100.

10. The skin protection device of claim 6, wherein the flexible body is stored on the backing sheet and can be peeled from the backing sheet prior to application on the body of the user.

11. A skin protection device comprising:
a flexible body comprised of a top surface having a plurality of perforations and a skin-safe, waterproof, adhesive bottom surface that can be applied to a body of a user; and
a biodegradable backing sheet that can receive and store the flexible body when the flexible body is in use.

12. The skin protection device of claim 11, wherein the top surface is comprised of a non-woven, random-spun synthetic fiber fabric material capable of blocking harmful ultra-violet (UV) radiation.

13. The skin protection device of claim 12, wherein the top surface matches a skin shade of the user.

14. The skin protection device of claim 11, wherein the flexible body can be applied to the body of the user in at least one of a nose area, a face area, a shoulder area, a chest area, an arm area, a leg area, a feet area, a hand area, a back area or a stomach area.

15. The skin protection device of claim 11, wherein the flexible body is shaped to mimic a shape of a body part that the skin protection device is applied to.

16. The skin protection device of claim 11, wherein the flexible body is infused with a plurality of titanium dioxide, zinc oxide or zinc particles to protect the body against a UV exposure.

17. The skin protection device of claim 11, wherein the flexible body is comprised of a SPF protection.

18. The skin protection device of claim 11, wherein the flexible body is peeled from the biodegradable backing sheet prior to application on the body of the user.

19. The skin protection device of claim 11, wherein the plurality of perforations are circular and aid in breathability of the flexible body.

20. The skin protection device of claim 11, wherein the plurality of perforations are linear and allow a plurality of skin protection devices to attach to one another and then be torn apart when ready for use.
